(19) United States
(12) Patent Application Publication
(10) Pub. No.: US 2025/0250295 A1
(43) Pub. Date: Aug. 7, 2025

(54) PROCESS FOR THE PREPARATION OF LOW-COLOR ALKYL POLYGLYCOSIDES, INVOLVING NEUTRALIZATION OF THE REACTION MEDIUM AFTER REMOVAL OF THE SUGAR

(71) Applicant: SOCIÉTÉ D'EXPLOITATION DE PRODUITS POUR LES INDUSTRIES CHIMIQUES SEPPIC, PARIS CEDEX 07 (FR)

(72) Inventors: Estelle ILLOUS, CASTRES (FR); Stéphane DESSILLA, CASTRES (FR)

(21) Appl. No.: 18/700,069

(22) PCT Filed: Oct. 12, 2022
(86) PCT No.: PCT/EP2022/078401

§ 371 Date: Apr. 10, 2024

(30) Foreign Application Priority Data
Oct. 13, 2021 (FR) 2110845

(51) Int. Cl. C07H 15/04

(52) U.S. Cl. CPC C07H 15/04

(57) ABSTRACT

Disclosed is a process for preparing alkyl polyglycosides having a color of less than or equal to 1.5 VCS, the process successively involving: a) a glycosylation step a) consisting in a reaction between at least one alcohol of formula (I) and at least one reducing sugar of formula (III): H—HO—O-(G)-H, in the presence of at least one acid catalyst (CA) at a minimum temperature of 100° C. and a maximum temperature of 120° C., b) a step b) of removing the reducing sugar of formula (III): H—O-(G)-H, which has not reacted in step a), from the reaction medium, c) a step c) of neutralizing the reaction medium from step b) with an aqueous solution comprising a basic agent (Ab), d) a step d) of recovering at least one composition (C) having a color less than or equal to 1.5 VCS.

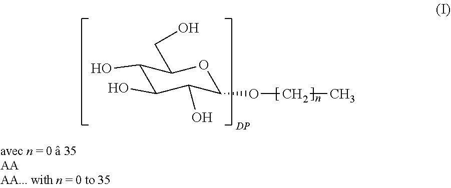

The present invention relates to a process for preparing low-color alkyl polyglycosides (color of less than 1.5 VCS) involving carbonate neutralizing agents. Alkyl polyglycosides or APGs are probably the best examples of biobased surfactants available on the market today. Their molecular structures are characterized by the simultaneous presence of a hydrophilic head derived from reducing sugars (D-glucose, D-xylose or D-rhamnose are the reducing sugars mainly available on the market on an industrial scale) and a lipophilic hydrocarbon chain of varying length (cf. formula I: simplified structure of an APG).

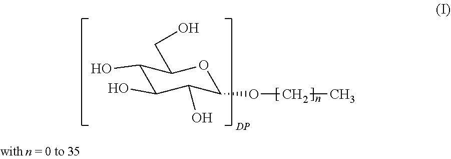

The process for the industrial scale manufacture thereof is relatively simple and uses, as raw materials: i) crystalline glucose, xylose or rhamnose resulting respectively from the complete hydrolysis of wheat, corn or potato starch or from the hydrolysis of wood hemicelluloses and ii) fatty alcohols from the oleochemical industry (hydrogenation of methyl esters resulting from the transesterification of plant triglycerides). Fischer glycosylation reactions then consist in linking these two raw materials together by creating a covalent chemical bond, like for example in reaction (II) between glucose and an alcohol.

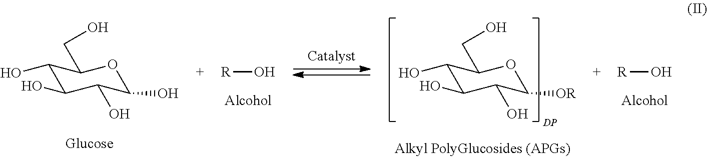

To carry out this glycosylation reaction, an acid catalyst of mineral or organic origin is necessary and an excess of alcohols is systematically introduced, thus acting as reactant and solvent. At the end of the reaction, the APGs are dispersed or solubilized in the excess alcohol which has not reacted.

The APGs are distinguished by the nature of the hydrocarbon alkyl chain R and by their average degree of polymerization DP of greater than 1 but less than or equal to 2.5.

At the end of the glycolysation reaction phase, a neutralization step is carried out in order to deactivate the catalyst and stop the reaction.

Depending on the length of the alkyl chain of the alcohol and the associated use, said alcohol is either removed or retained.

The neutralization step differs depending on the length of the hydrocarbon alkyl chain. In the case where the latter has a number of carbon atoms of less than 12, the neutralization is carried out by an aqueous solution of sodium hydroxide. The excess fatty alcohols present at the end of glycosylation are then removed by high vacuum distillation or molecular distillation, or by evaporation, generally, using a falling-film thin-film evaporator or a short-path thin-film evaporator, and the APG concentrate collected is finally dissolved in water. The commercial products thus obtained are therefore in the form of aqueous APG solutions with a weight concentration of between 40% and 80%.

In the case where the hydrocarbon alkyl chain R has a number of carbon atoms of greater than or equal to 12, the neutralization is generally carried out by sodium hydroxide or by potassium hydroxide, alone or in combination with a reducing agent such as in the European patent published under number EP 0 077 167, in the European patent application published under number EP 0 338 151 A1, in the European patent EP 0 388 857 B1, such as for example sodium borohydride (NaBH₄) or sodium hypophosphite (NaH₂PO₂). The mixture of APG and excess fatty alcohols is isolated after neutralization and is sold as is. The weight proportion of APG and fatty alcohols depends directly on the molar stoichiometry adopted at the start for the raw materials and on their reactivity. However, proportions of from 5% to 30% by weight of APG and from 70% to 95% of fatty alcohols are generally observed. The corresponding products are therefore solid in the form of flakes or beads, or in a liquid form, depending on the nature of the hydrocarbon alkyl chain R.

However, the step of neutralizing emulsifying APGs having a hydrocarbon alkyl chain R that contains a number of carbon atoms of greater than or equal to 12 with a base from the prior art (e.g. NaOH, KOH), in order to achieve a pH value of a 5 wt % dispersion in water of the neutralized medium of between 5.5 and 7.5, gives rise to significant coloration of the product.

For the purposes of the present invention, “measurement of the pH of a 5 wt % dispersion in water” denotes the analytical method for measuring the pH of a dispersion of an APG-based composition according to the provisions of NF EN 1262, said measurement is carried out by potentiometric measurement using a combined pH electrode (aqueous media) and a pH meter.

This coloration may impair the organoleptic qualities of the finished products into which the APG-containing compositions are introduced. For this reason, solutions are provided to minimize the coloration of compositions containing APGs having a hydrocarbon chain R that contains a number of carbon atoms of greater than or equal to 12. Two levers known from the prior art are conventionally used to obtain such low-color (<1.5 VCS) compositions based on APGs having a hydrocarbon alkyl chain R that contains a number of carbon atoms of greater than or equal to 12.

For the purposes of the present invention, a “low-color composition” denotes a composition for which the Gardner color scale, as defined by DIN-ISO 4630, is less than or equal to 1.5 VCS. The Gardner color scale is measured using a LICO 200/Dr LANGE (or equivalent) colorimeter that performs light transmission measurements on any medium. Such a colorimeter operates with a halogen lamp corresponding to the standard illuminant C, defined by DIN 5033 and with a 2° standard observer. During the measurement, a reference radiation beam compensates for variations in the recorded values due to lamp and temperature differences.

The first lever consists in combining a reducing agent with the base used. Among these reducing agents, mention may be made of sodium borohydride (NaBH₄) or of sodium hypophosphite (NaH₂PO₂). This solution is not entirely satisfactory. Specifically, although very effective in minimizing the coloration of the treated composition, NaBH₄ is a reducing agent which is dangerous to handle and to use (corrosive product, release of hydrogen). NaH₂PO₂ is itself not very effective, even if it is introduced at high concentration.

The second lever commonly used and described in the prior art, for minimizing the color of compositions based on APGs having a hydrocarbon alkyl chain R that contains a number of carbon atoms of greater than or equal to 12, is carrying out a decolorization with hydrogen peroxide (H₂O₂) during the finishing step. Although effective, this step is however tedious because it requires the pH value of a 5 wt % dispersion in water to be adjusted to between 7.0 and 7.5 while maintaining the oxidizing power of the medium by adding H₂O₂. This step, which is difficult to carry out, can last several hours and therefore significantly increase the production time, reducing productivity.

The technical problem to be solved is therefore to find an alternative to the neutralization of compositions based on APGs having a hydrocarbon alkyl chain R that contains a number of carbon atoms of greater than or equal to 12. This alternative must be effective and easy to implement, while guaranteeing a color of less than or equal to 1.5 VCS without any decolorization step.

One solution of the present invention is a process for preparing a composition (C) having a color of less than or equal to 1.5 VCS, comprising, for 100% of its weight:
(i) an amount of greater than or equal to 40% by weight and less than or equal to 95% by weight, preferably greater than or equal to 50% by weight and less than or equal to 95% by weight, even more preferentially greater than or equal to 70% by weight and less than or equal to 90% by weight of an alcohol of formula (I):
   R—OH (I), wherein R represents a linear or branched, saturated or unsaturated hydrocarbon radical, which may include at least one hydroxyl function, and including from 12 to 22 carbon atoms or a mixture of alcohols of formula (I);
(ii) an amount of greater than or equal to 5% by weight and less than or equal to 60% by weight, preferably greater than or equal to 5% by weight and less than or equal to 50% by weight, and even more preferentially greater than or equal to 10% and less than or equal to 30% by weight of a composition (Cl) represented by formula (11):
   R—O-(G)x-H (II), wherein the residue G represents the residue of a reducing sugar, R represents a radical as defined in formula (I) and x, which indicates the average degree of polymerization of the residue G represents a decimal number greater than 1.05 and less than or equal to 2.5, or a mixture of compositions (Cl) of formula (II); it being understood that the sum of the weight proportions of the compounds and compositions (I) and (II) is equal to 100% by weight,
said process successively comprising:
a) a step a) of glycosylation, consisting of a reaction between at least one alcohol of formula (I) and at least one reducing sugar of formula (III): H—O-(G)-H (III), in the presence of at least one acid catalyst (CA), at a temperature above or equal to 100° C. and below or equal to 120° C., preferably above or equal to 100° C. and below or equal to 115° C., even more preferentially above or equal to 100° C. and below or equal to 110° C.,
   the at least one acid catalyst (CA) being selected from the members of the group consisting of sulfuric acid, hydrochloric acid, phosphoric acid, nitric acid, hypophosphorous acid, methanesulfonic acid, para-toluenesulfonic acid, trifluoromethanesulfonic acid and acid ion-exchange resins,
b) a step b) of removing the reducing sugar of formula (11), which has not reacted in step a), from the reaction medium,
c) a step c) of neutralizing the reaction medium from step b) with an aqueous solution comprising a basic agent (Ab) selected from the members of the group consisting of:
   carbonates of formula (IVa):

XnCO₃ (IVa),
wherein X represents a sodium or potassium atom and n is an integer equal to 2, or X represents a calcium atom or a magnesium atom and n is an integer equal to 1, or
hydrogen carbonates of formula (IVb):

Y(HCO₃)m (IVb),
wherein Y represents a sodium or potassium atom and m is an integer equal to 1, or Y represents a calcium atom or a magnesium atom and m is an integer equal to 2,
the neutralization being carried out so as to obtain a reaction medium for which the 5 wt % dispersion of said reaction medium in water has a pH value of between 5.5 and 7.5, and
d) a step d) of recovering at least one composition (C) having a color of less than or equal to 1.5 VCS.

The color scale characterizing the composition (C) prepared according to the process which is the subject of the present invention is the Gardner color scale, as defined by the DIN-ISO 463 standard. The Gardner color scale is measured using a LICO 200/Dr LANGE (or equivalent) colorimeter that performs light transmission measurements on any medium. Such a colorimeter operates with a halogen lamp corresponding to the standard illuminant C, defined by DIN 5033 and with a 2° standard observer. During the measurement, a reference radiation beam compensates for variations in the recorded values due to lamp and temperature differences.

The unit of expressing the Gardner color scale, characterizing the composition (C) prepared according to the process which is the subject of the present invention, is VCS.

Depending on the case, the process according to the invention may have one or more of the following characteristics:
said composition (Cl) consists of a mixture of compounds represented by the formulae (II1), (II2), (II3), (II4) and (II5):

R—O-(G)1-H (II1),

R—O-(G)2-H (II2),

R—O-(G)3-H (II3),

R—O-(G)4-H (II4),

R—O-(G)5-H (II5),
in the respective molar proportions a1, a2, a3, a4 and a5, such that:
   the sum: a1+a2+a3+a4+a5 is equal to 1, and
   the sum a1+2a2+3a3+4a4+5a5 is equal to x;
the composition (C) comprises an amount of less than or equal to 2% by weight, preferably less than or equal to 1% by weight, of the reducing sugar of formula (III):

H—O-(G)-H (III);
it being understood that the sum of the weight proportions of the compounds and compositions (I), (II) and (III) is equal to 100% by weight;
the basic agent (Ab) present in the aqueous solution is sodium carbonate of formula (IVa) wherein X represents a sodium atom and n is equal to 2;
in step c) the aqueous solution of the basic agent (Ab) comprises between 10% and 25% by weight of said basic agent (Ab);
step c) is carried out in two stages: a first stage during which neutralization is carried out so as to obtain a reaction medium for which a 5 wt % dispersion in water has a pH value of between 3.5 and 5.5, with a solution of sodium hydroxide (NaOH) or potassium hydroxide (KOH), then a second stage during which the neutralization is carried out so as to obtain a reaction medium for which a 5 wt % dispersion in water has a pH value of between 5.5 and 7.5, with an aqueous solution of basic agent (Ab);
the reducing sugar of formula (III) chosen for the glycolysation of step a) is chosen from the members of the group consisting of glucose, xylose, arabinose and rhamnose;
step b) of removing the reducing sugar of formula (III) is carried out by filtration, centrifugation or decantation;
step a) comprises the following successive substeps:
   i) introducing an alcohol of formula (I) into a reactor (Re) equipped with a mechanical stirrer and a vacuum device;
   ii) heating the alcohol of formula (I) to a temperature of between 80° C. and 90° C. under mechanical stirring;
   iii) charging the reducing sugar of formula (III) to the reactor (Re);
   iv) introducing at least one catalyst (CA) into the reactor (Re);
   v) heating, under vacuum, the reaction medium present in the reactor (Re) to a temperature of between 100° C. and 110° C. for the duration of the reaction, and
   vi) cooling the medium from substep v) to a temperature of between 70° C. and 80° C.;
in formula (I) and/or in formula (II) the radical R is chosen from the following radicals: lauryl (or n-dodecyl), myristyl (or n-tetradecyl), n-pentadecyl, cetyl (or n-hexadecyl), n-heptadecyl, stearyl (or n-octadecyl), palmitoleyl (or 9-hexadecenyl), oleyl (or 9-octadecenyl), linoleyl (9,12-octadecadienyl), linolenyl (or 6,9,12-octadecatrienyl), nonadecyl, arachidyl (or n-eicosyl), behenyl (or n-docosyl), erucyl (13-docosenyl), or 12-hydroxystearyl;
the radical R is chosen from the following radicals: 2-hexyloctyl, 2-hexyldecyl, 2-hexyldodecyl, 2-octyldecyl, 2-octyldodecyl, 2-decyltetradecyl, isostearyl (or 16-methylheptadecyl) or isomyristyl (or 13-methyltridecyl);
during substeps ii) to iv) the reactor (Re) is inerted under nitrogen;
the process comprises, between substeps ii) and iii), a vacuum step, preferably at a pressure of less than or equal to 50 millibar;
substep vi) is carried out at atmospheric pressure.

The use of carbonates of formula (IVa) or hydrogen carbonates of formula (IVb) does not contribute to increasing the color of the composition (C) (the presence of a reducing agent then not being necessary) while neutralizing to the desired pH a 5 wt % dispersion of the composition (C) (the value of which is between 5.5 and 7.5). The use of a basic agent (Ab) makes it possible to avoid a decolorization step involving the use of a peroxide agent, or the like, since it makes it possible to achieve a color less than or equal to 1.5 VCS.

The term “reducing sugar” in the definition of formula (II) and in the definition of formula (III) denotes saccharide derivatives that do not have in their structures any glycoside bonds established between an anomeric carbon and the oxygen of an acetal group as defined in the reference publication: “Biochemistry”, Daniel Voet/Judith G. Voet, page 250, John Wiley & Sons, 1990.

The oligomeric structure (G)x present in formula (II) may be in any isomeric form, whether this relates to optical isomerism, geometrical isomerism or regioisomerism; it may also represent a mixture of isomers.

In formula (II) as defined above, the radical R is bonded to G via the anomeric carbon of the saccharide residue, so as to form an acetal function.

According to a particular aspect of the present invention, in the definition of the compounds of formulae (II) and of formula (III), G represents the residue of a reducing sugar chosen from glucose, dextrose, sucrose, fructose, idose, gulose, galactose, maltose, isomaltose, maltotriose, lactose, cellobiose, mannose, ribose, xylose, arabinose, lyxose, allose, altrose, rhamnose, dextran or tallose.

According to a particular aspect of the present invention, in the definition of the compounds of formula (II), G represents the residue of a reducing sugar chosen from the residues of glucose, xylose, arabinose or rhamnose, and x represents a decimal number greater than or equal to 1.05 and less than or equal to 2.5.

According to an even more particular aspect of the present invention, in the definition of the compounds of formula (II), G represents the residue of a reducing sugar chosen from the residues of glucose, xylose, arabinose or rhamnose, and x represents a decimal number greater than or equal to 1.05 and less than or equal to 2.0, and even more particularly greater than or equal to 1.25 and less than or equal to 2.0.

According to another particular aspect of the present invention, the reducing sugar of formula (III) is chosen from the members of the group consisting of glucose, dextrose, sucrose, fructose, idose, gulose, galactose, maltose, isomaltose, maltotriose, lactose, cellobiose, mannose, ribose, xylose, arabinose, lyxose, allose, altrose, rhamnose, dextran or tallose.

According to a more particular aspect of the present invention, the reducing sugar of formula (III) is chosen from glucose, xylose, arabinose or rhamnose.

The process according to the invention consists in carrying out the filtration/centrifugation step before carrying out the neutralization of the product with an aqueous solution comprising a basic agent (Ab).

According to a particular aspect, one subject of the process is the preparation of a composition (C) having a color of less than or equal to 1.5 VCS, comprising, for 100% of its weight:
from 45% to 55% by weight of a mixture (M1) of alcohols of formula (I) comprising, for 100% of the weight of said mixture (M1), 50% by weight of an alcohol of formula (I) wherein R represents the n-hexadecyl radical and 50% by weight of an alcohol of formula (I) wherein R represents the n-octadecyl radical,
from 45% to 54% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical,
less than 1% by weight of glucose.

According to a particular aspect, one subject of the process is the preparation of a composition (C) having a color of less than or equal to 1.5 VCS, comprising, for 100% of its weight:
from 45% to 55% by weight of a mixture (M′1) of alcohols of formula (I) comprising, for 100% of the weight of said mixture (M′1), 70% by weight of an alcohol of formula (I) wherein R represents the n-hexadecyl radical and 30% by weight of an alcohol of formula (I) wherein R represents the n-octadecyl radical,
from 45% to 54% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical,
less than 1% by weight of glucose.

According to a particular aspect, one subject of the process is the preparation of a composition (C) having a color of less than or equal to 1.5 VCS, comprising, for 100% of its weight:
from 75% to 90% by weight of a mixture (M″1) of alcohols of formula (I) comprising, for 100% of the weight of said mixture (M″1), 50% by weight of an alcohol of formula (I) wherein R represents the n-hexadecyl radical and 50% by weight of an alcohol of formula (I) wherein R represents the n-octadecyl radical,
from 10% to 24% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical,
less than 1% by weight of glucose.

According to a particular aspect, one subject of the process is the preparation of a composition (C) having a color of less than or equal to 1.5 VCS, comprising, for 100% of its weight:
from 75% to 90% by weight of a mixture (M′″1) of alcohols of formula (I) comprising, for 100% of the weight of said mixture (M′″1), 70% by weight of an alcohol of formula (I) wherein R represents the n-hexadecyl radical and 30% by weight of an alcohol of formula (I) wherein R represents the n-octadecyl radical,
from 10% to 24% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-hexadecyl radical and the n-octadecyl radical,
less than 1% by weight of glucose.

According to a particular aspect, one subject of the process is the preparation of a composition (C) having a color of less than or equal to 1.5 VCS, comprising, for 100% of its weight:
from 75% to 90% by weight of an alcohol of formula (I) wherein R represents the n-tetradecyl radical
from 10% to 24% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-tetradecyl radical,
less than 1% by weight of glucose.

According to a particular aspect, one subject of the process is the preparation of a composition (C) having a color of less than or equal to 1.5 VCS, comprising, for 100% of its weight:
from 75% to 90% by weight of a mixture of alcohols of formula (I) wherein R represents the n-dodecyl radical, n-tetradecyl radical, n-hexadecyl radical and n-octadecyl radical
from 10% to 24% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-dodecyl radical, the n-tetradecyl radical, the n-hexadecyl radical and the n-octadecyl radical,
less than 1% by weight of glucose.

According to a particular aspect, one subject of the process is the preparation of a composition (C) having a color of less than or equal to 1.5 VCS, comprising, for 100% of its weight:
from 75% to 90% by weight of a mixture of alcohols of formula (I) wherein R represents the n-eicosyl radical and the n-docosyl radical
from 10% to 24% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-eicosyl radical and the n-docosyl radical,
less than 1% by weight of glucose.

According to a particular aspect, one subject of the process is the preparation of a composition (C) having a color of less than or equal to 1.5 VCS, comprising, for 100% of its weight:
from 75% to 90% by weight of a mixture of alcohols of formula (I) wherein R represents the n-dodecyl radical, the n-tetradecyl radical, the n-hexadecyl radical, the n-eicosyl radical and the n-docosyl radical,
from 10% to 24% by weight of at least one composition (Cl) represented by formula (II) wherein G represents the glucosyl or α,β-D-glucopyranosyl radical, obtained from the removal of the hemiacetal hydroxyl group of α,β-D-glucopyranose, x represents a decimal number of greater than or equal to 1.05 and less than or equal to 2.0, R represents the n-dodecyl radical, the n-tetradecyl radical, the n-hexadecyl radical, the n-eicosyl radical and the n-docosyl radical,
less than 1% by weight of glucose.

According to a particular aspect, one subject of the process is the preparation of a composition (C) having a color of less than or equal to 1.5 VCS, comprising, for 100% of its weight:
from 70% to 90% by weight of a mixture of alcohols of formula (I) wherein R represents the n-dodecyl radical, the n-tetradecyl radical, the n-hexadecyl radical, the n-eicosyl radical and the n-docosyl radical,
from 10% to 29% by weight of a composition (Cl) represented by formula (II) wherein G represents the xylosyl or α,β-D-xylopyranosyl radical, obtained from the removal of the hemiacetal hydroxyl group of α,β-D-xylopyranose, x represents a decimal number of greater than or equal to 1.05 and less than or equal to 2.0, R represents the 2-octyldodecyl radical,
less than 1% by weight of xylose.

According to a particular aspect, the basic agent (Ab) present in the aqueous solution is potassium carbonate of formula (IVa) wherein X represents the potassium atom and n is equal to 2.

According to a particular aspect, the basic agent (Ab) present in the aqueous solution is sodium hydrogen carbonate of formula (IVb) wherein Y represents a sodium atom and m is equal to 1.

According to a particular aspect, the acid catalyst (CA) is chosen from the members of the group consisting of sulfuric acid, phosphoric acid, hypophosphorous acid, methanesulfonic acid and p-toluenesulfonic acid.

### EXAMPLES

A. Comparison of the Effect of the Basic Agent (Ab) on the Color of a Composition of Fatty Alcohols and Alkyl Polyglucosides when the Neutralizing Basic Agent (Ab) Used is a Carbonate According to the Invention or Sodium Hydroxide (Comparative Neutralizing Agent).

Comparisons between a carbonate and sodium hydroxide were made as a function of the pH range following the substep of filtration of the reaction medium.

Table 1 below collates the neutralization results involving the filtered reaction medium obtained from glycosylation reactions between crystalline glucose and various fatty alcohols in the form of cuts or in pure form: C-16/18-cetearyl cut, 1-tetradecanol (C14 alcohol), 1-dodecanol (C12 alcohol). The various parameters studied are the nature of the pre-neutralizing agent (pH value between 3.5 and 5.5 of the 5 wt % dispersion in water of the post-filtration and neutralization medium, pH value between 5.5 and 7.5 of the 5 wt % dispersion in water of the medium (Na₂CO₃ and/or NaOH).

### 1. Examples According to the Invention

### Example 1.1 Cut of 16/18 Alcohols and Na₂CO₃ as Neutralizing Agent According to the Invention

### Step 1: Glycosylation Reaction:

529.3 g of cetearyl alcohol (C-16/18) are charged to a reactor equipped with a mechanical stirrer and a vacuum distillation setup. The alcohol is melted at 85° C., stirred and sparged with nitrogen. The medium is placed under vacuum at pressures of less than 50 millibar. 69.5 g of anhydrous glucose in powder form are added. The medium is inerted under nitrogen. To start the etherification (glycosylation) reaction, 0.5 g of a 50% aqueous solution of hypophosphorous acid (H₃PO₂) and then 0.6 g of a 98% aqueous solution of sulfuric acid (H₂SO₄) are added and the temperature is increased and maintained at 105° C. The reaction is continued for 5 hours 45 minutes.

### Step 2: Filtration and Neutralization of the Reaction Medium:

The medium is cooled to 80° C. at atmospheric pressure and then filtered through a bell-shaped filter equipped with a plate (3-6 m) in order to remove the residual sugar. 167 g of the product thus obtained are then reintroduced into a reactor at 85° C. and then neutralized by adding, with stirring, 1.7 g of a 10% aqueous solution of Na₂CO₃ in order to obtain a composition referenced (Composition 1).

### Analyses:

the pH value of a 5 wt % dispersion in water of (Composition 1) is 6.4, and
the color measurement of (Composition 1) is 0.9 VCS.

### Example 1.2 Cut of 16/18 Alcohols and NaOH as Pre-Neutralizing Agent and Na₂CO₃ as Neutralizing Agent According to the Invention

### Step 1: Glycosylation Reaction:

529.3 g of cetearyl alcohol (C-16/18) are charged to a reactor equipped with a mechanical stirrer and a vacuum distillation setup. The alcohol is melted at 85° C., stirred and sparged with nitrogen. The medium is placed under vacuum at pressures of less than 50 millibar. 69.5 g of anhydrous glucose in powder form are added. The medium is inerted under nitrogen. To start the etherification (glycosylation) reaction, 0.5 g of a 50% aqueous solution of H₃PO₂ and then 0.6 g of a 98% aqueous solution of H₂SO₄ are added and the temperature is increased and maintained at 105° C. The reaction is continued for 5 hours 45 minutes.

### Step 2: Filtration and Neutralization of the Reaction Medium:

The medium is cooled to 80° C. at atmospheric pressure and then filtered through a bag filter (100 μm) in order to remove the residual sugar. 264.4 g of the product thus obtained are then reintroduced into a reactor at 75° C. and then pre-neutralized by introducing, with stirring, 0.56 g of a 25% aqueous solution of NaOH in order to achieve a pH value equal to 4.1 for a 5 wt % dispersion in water of the medium present in the reactor and a color of 1.6 VCS.

The product is then neutralized, still with stirring, at 75° C. by introducing 0.9 g of a 10% aqueous solution of Na₂CO₃ in order to obtain a composition referenced (Composition 2).

### Analyses:

the pH value of a 5 wt % dispersion in water of (Composition 2) is 6.4, and
the color measurement of (Composition 2) is 1.0 VCS.

### Example 1.3 1-Tetradecanol (Myristyl Alcohol) and NaOH as Pre-Neutralizing Agent and Na₂CO₃ as Neutralizing Agent According to the Invention

### Step 1: Glycosylation Reaction:

355.8 g of myristyl alcohol (1-tetradecanol) are charged to a reactor equipped with a mechanical stirrer and a vacuum distillation setup. The myristyl alcohol is melted at 85° C., stirred and sparged with nitrogen. The medium is placed under vacuum at 25 millibar. 49.7 g of anhydrous glucose in powder form are added. The medium is inerted under nitrogen. To start the etherification (glycosylation) reaction, 0.38 g of a 50% aqueous solution of H₃PO₂ and then 0.61 g of a 98% aqueous solution of H₂SO₄ are added and the temperature is increased and maintained at 105° C. The reaction is continued for 5 hours.

### Step 2: Neutralization of the Reaction Medium:

The medium is cooled to 80° C. at atmospheric pressure and then filtered through a K1 filter (4 μm) in order to remove the residual sugar. 164 g of the product thus obtained are then reintroduced into a reactor at 80° C. and then pre-neutralized by introducing 0.46 g of a 25% aqueous solution of NaOH, with stirring, in order to achieve a pH value of 6.0 for a 5 wt % dispersion in water of the medium present in the reactor.

The product is finally neutralized at 80° C. in a reactor with stirring, by introducing 0.29 g of a 10% aqueous solution of Na₂CO₃ in order to obtain a composition referenced (Composition 3).

### Analyses:

the pH value of a 5 wt % dispersion in water of (Composition 3) is 6.6, and
the color measurement of (Composition 3) is 1.1 VCS.

### Example 1.4 1-Tetradecanol (Myristyl Alcohol) and Na₂CO₃ as Neutralizing Agent According to the Invention

### Step 1: Glycosylation Reaction:

355.8 g of myristyl alcohol (1-tetradecanol) are charged to a reactor equipped with a mechanical stirrer and a vacuum distillation setup. The alcohol is melted at 85° C., stirred and sparged with nitrogen. The medium is placed under vacuum at 25 millibar. 49.7 g of anhydrous glucose in powder form are added. The medium is inerted under nitrogen. To start the etherification (glycosylation) reaction, 0.38 g of a 50% aqueous solution of H₃PO₂ and then 0.61 g of a 98% aqueous solution of H₂SO₄ are added and the temperature is increased and maintained at 105° C. The reaction is continued for 5 hours.

### Step 2: Neutralization of the Reaction Medium:

The medium is cooled to 80° C. at atmospheric pressure and then filtered through a K1 filter (4 μm) in order to remove the residual sugar. 160.5 g of the product are then reintroduced into a reactor at 70° C. and then neutralized by adding, with stirring, 1.77 g of a 10% aqueous solution of Na₂CO₃ in order to obtain a composition referenced (Composition 4).

### Analyses:

the pH value of a 5 wt % dispersion in water of (Composition 4) is 7.4, and
the color measurement of (Composition 4) is 1.0 VCS.

### Example 1.5 1-Dodecanol (Lauryl Alcohol) and Na₂CO₃ as Neutralizing Agent According to the Invention

### Step 1: Glycosylation Reaction:

190.6 g of lauryl alcohol (1-dodecanol) are charged to a reactor equipped with a mechanical stirrer and a vacuum distillation setup. The alcohol is melted at 85° C., stirred and sparged with nitrogen. The medium is placed under vacuum at 30 millibar. 26.6 g of anhydrous glucose in powder form are added. The medium is inerted under nitrogen. To start the etherification (glycosylation) reaction, 0.2 g of a 50% aqueous solution of H₃PO₂ and then 0.3 g of a 98% aqueous solution of H₂SO₄ are added and the temperature is increased and maintained at 105° C. The reaction is continued for 5 hours.

### Step 2: Neutralization of the Reaction Medium:

The medium is cooled to 70° C. at atmospheric pressure and then filtered through a K1 filter (4 μm) in order to remove the residual sugar. The product (160 g) is then reintroduced into a reactor at 75° C. and then neutralized by introducing, with stirring, 1.77 g of a 10% aqueous solution of Na₂CO₃ in order to obtain a composition referenced (Composition 5).

### Analyses:

the pH value of a 5 wt % dispersion in water of (Composition 5) is 6.9, and
the color measurement of (Composition 5) is 1.0 VCS.

### 2. Comparative Examples

### Example 2.1: Comparative Example (Cut of 16/18 Alcohols and NaOH as Pre-Neutralizing Agent and NaOH as Neutralizing Agent)

### Step 1: Glycosylation Reaction:

529.3 g of cetearyl alcohol (C-16/18) are charged to a reactor equipped with a mechanical stirrer and a vacuum distillation setup. The alcohol is melted at 85° C., stirred and sparged with nitrogen. The medium is placed under vacuum at pressures of less than 50 millibar. 69.5 g of anhydrous glucose in powder form are added. The medium is inerted under nitrogen. To start the etherification (glycosylation) reaction, 0.5 g of a 50% aqueous solution of H₃PO₂ and then 0.6 g of a 98% aqueous solution of H₂SO₄ are added and the temperature is increased and maintained at 105° C. The reaction is continued for 5 hours 45 minutes.

### Step 2: Neutralization of the Reaction Medium:

The medium is cooled to 80° C. at atmospheric pressure and then filtered through a bell-shaped filter equipped with a plate (3-6 am) in order to remove the residual sugar. 500 g of the product are then reintroduced into a reactor at 85° C. and then neutralized by adding, with stirring, 0.81 g of a 40% aqueous solution of NaOH in order to obtain a composition referenced (Composition 1′).

### Analyses:

the pH value of a 5 wt % dispersion in water of (Composition 1′) is 6.5, and
the color measurement of (Composition 1′) is 2.8 VCS.

TABLE 1

| | Composition | Composition | Composition | Composition | 5898 JG2 | |
|---|---|---|---|---|---|---|
| Reference | 1′ | 1 | 2 | 3 | Composition 4 | 5918 JG |
| APG alkyl chain | C-16/18 | | | C-14 | | C-12 |
| Pre-neutralizing | NaOH | Na₂CO₃ | NaOH | NaOH | Na₂CO₃ | Na₂CO₃ |
| basic agent | | | | | | |
| Neutralizing | NaOH | Na₂CO₃ | Na₂CO₃ | Na₂CO₃ | Na₂CO₃ | Na₂CO₃ |
| basic agent | | | | | | |
| pH of 5% | 6.5 | 6.4 | 6.4 | 6.6 | 7.4 | 6.9 |
| dispersion | | | | | | |
| in water | | | | | | |
| Color (VCS) | 2.8 | 0.9 | 1.0 | 1.1 | 1.0 | 1.0 |

These tests demonstrate that:
neutralization to a pH of the 5% dispersion in water of between 5.5 and 7.5 by sodium carbonate alone makes it possible to achieve the desired level of coloration (less than or equal to 1.5 VCS for (Composition 1)) whereas neutralization to a pH of the 5% dispersion in water of between 5.5 and 7.5 by sodium hydroxide alone results in a coloration of greater than 1.5 VCS, in this case up to about 2.8 VCS (Composition 1′).
when the pre-neutralization step is carried out with a solution of NaOH or Na₂CO₃ and the neutralization step is carried out with an aqueous solution of Na₂CO₃, the colors of the products obtained are very low (less than or equal to 1.5 VCS).

It has therefore been shown that it is possible to neutralize the compositions obtained by carrying out the process according to the invention with a solution of Na₂CO₃, in a pH range of between 5.5 and 7.5 for a 5% dispersion in water of said compositions, after filtration of the residual sugars without increasing the color of the compositions to a value greater than 1.5 VCS.

1. A process for preparing a composition (C) having a color of less than or equal to 1.5 VCS, comprising, for 100% of its weight:
(i) an amount of greater than or equal to 40% by weight and less than or equal to 95% by weight of an alcohol of formula (I):
R—OH (I), wherein R represents a linear or branched, saturated or unsaturated hydrocarbon radical, which may include at least one hydroxyl function, and including from 12 to 22 carbon atoms or a mixture of alcohols of formula (I);
(ii) an amount of greater than or equal to 5% by weight and less than or equal to 60% by weight, of a composition (Cl) represented by formula (II):
R—O-(G)x-H (II), wherein the residue G represents the residue of a reducing sugar, R represents a radical as defined in formula (I) and x, which indicates the average degree of polymerization of the residue G represents a decimal number greater than 1.05 and less than or equal to 2.5, or a mixture of compositions (Cl) of formula (II);
it being understood that the sum of the weight proportions of the compounds and compositions (I) and (II) is equal to 100% by weight,
said process successively comprising:
a) a step a) of glycosylation, consisting of a reaction between at least one alcohol of formula (I) and at least one reducing sugar of formula (III): H—O-(G)-H (Ill), in the presence of at least one acid catalyst (CA), at a temperature above or equal to 100° C. and below or equal to 120° C., the at least one acid catalyst (CA) being selected from members of the group consisting of sulfuric acid, hydrochloric acid, phosphoric acid, nitric acid, hypophosphorous acid, methanesulfonic acid, para-toluenesulfonic acid, trifluoromethanesulfonic acid and acid ion-exchange resins,
b) a step b) of removing the reducing sugar of formula (III), which has not reacted in step a), from the reaction medium,
c) a step c) of neutralizing the reaction medium from step b) with an aqueous solution comprising a basic agent (Ab), the basic agent (Ab) being selected from the members of the group consisting of:
carbonates of formula (IVa):
XnCO₃ (IVa),
wherein X represents a sodium or potassium atom and n is an integer equal to 2, or X represents a calcium atom or a magnesium atom and n is an integer equal to 1, or
hydrogen carbonates of formula (IVb):
Y(HCO₃)m (IVb),
wherein Y represents a sodium or potassium atom and m is an integer equal to 1, or Y represents a calcium atom or a magnesium atom and m is an integer equal to 2,
the neutralization being carried out so as to obtain a reaction medium for which the 5 wt % dispersion of said reaction medium in water has a pH value of between 5.5 and 7.5, and
d) a step d) of recovering at least one composition (C) having a color of less than or equal to 1.5 VCS.

2. The process as claimed in claim 1, wherein said composition (C1) consists of a mixture of compounds represented by formulae (II1), (II2), (II3), (II4) and (II5):
R—O-(G)1-H (II1),
R—O-(G)2-H (II2),
R—O-(G)3-H (II3),
R—O-(G)4-H (II4),
R—O-(G)5-H (II5),
in the respective molar proportions a1, a2, a3, a4 and a5, such that:
the sum: a1+a2+a3+a4+a5 is equal to 1, and
the sum a1+2a2+3a3+4a4+5a5 is equal to x.

3. The process as claimed in claim 1, wherein the composition (C) comprises an amount of less than or equal to 2% by weight, preferably less than or equal to 1% by weight, of the reducing sugar of formula (III):
H—O-(G)-H (III);
it being understood that the sum of the weight proportions of the compounds and compositions (I), (II) and (Ill) is equal to 100% by weight.

4. The process as claimed in claim 1, wherein the basic agent (Ab) present in the aqueous solution is sodium carbonate of formula (IVa) wherein X represents a sodium atom and n is equal to 2.

5. The process as claimed in claim 1, wherein in step c) the aqueous solution of the basic agent (Ab) comprises between 10% and 25% by weight of said basic agent (Ab).

6. The process as claimed in claim 1, wherein step c) is carried out in two stages: a first stage during which neutralization is carried out so as to obtain a reaction medium for which a 5 wt % dispersion in water has a pH value of between 3.5 and 5.5, with a solution of sodium hydroxide (NaOH) or potassium hydroxide (KOH), then a second stage during which the neutralization is carried out so as to obtain a reaction medium for which a 5 wt % dispersion in water has a pH value of between 5.5 and 7.5, with an aqueous solution of basic agent (Ab).

7. The process as claimed in claim 1, wherein the reducing sugar of formula (III) chosen for the glycolysation of step a) is chosen from the members of the group consisting of glucose, xylose, arabinose and rhamnose.

8. The process as claimed in claim 1, wherein step b) of removing the reducing sugar of formula (III) is carried out by filtration, centrifugation or decantation.

9. The process as claimed in claim 1, wherein step a) comprises the following successive substeps:
i) introducing an alcohol of formula (I) into a reactor (Re) equipped with a mechanical stirrer and a vacuum device;
ii) heating the alcohol of formula (I) to a temperature of between 80° C. and 90° C. under mechanical stirring;
iii) charging the reducing sugar of formula (III) to the reactor (Re);
iv) introducing at least one catalyst (CA) into the reactor (Re);
v) heating, under vacuum, the reaction medium present in the reactor (Re) to a temperature of between 100° C. and 110° C. for the duration of the reaction, and
vi) cooling the medium from substep v) to a temperature of between 70° C. and 80° C.

10. The process as claimed in claim 9, wherein in formula (I) and/or in formula (II) the radical R is chosen from the following radicals: lauryl (or n-dodecyl), myristyl (or n-tetradecyl), n-pentadecyl, cetyl (or n-hexadecyl), n-heptadecyl, stearyl (or n-octadecyl), palmitoleyl (or 9-hexadecenyl), oleyl (or 9-octadecenyl), linoleyl (9,12-octadecadienyl), linolenyl (or 6,9,12-octadecatrienyl), nonadecyl, arachidyl (or n-eicosyl), behenyl (or n-docosyl), erucyl (13-docosenyl), or 12-hydroxystearyl.

11. The process as claimed in claim 9, wherein in formula (I) and/or in formula (II) the radical R is chosen from the following radicals: 2-hexyloctyl, 2-hexyldecyl, 2-hexyldodecyl, 2-octyldecyl, 2-octyldodecyl, 2-decyltetradecyl, isostearyl (or 16-methylheptadecyl) or isomyristyl (or 13-methyltridecyl).

12. The process as claimed in claim 9, wherein during substeps ii) to iv), the reactor (Re) is inerted under nitrogen.

13. The process as claimed in claim 9, wherein it comprises, between substeps ii) and iii), a vacuum step, preferably at a pressure of less than or equal to 50 millibar.

14. The process as claimed in claim 9, wherein substep vi) is carried out at atmospheric pressure.
